(19) United States
(12) Patent Application Publication
(10) Pub. No.: US 2011/0033607 A1
(43) Pub. Date: Feb. 10, 2011

(54) AEROSOLIZED FINGERPRINT POWDER COMPOSITIONS

(75) Inventors: Phillip G. Pitts, Franklin, TN (US); James R. Vinson, Clarksville, TN (US)

Correspondence Address: STITES & HARBISON PLLC, 401 COMMERCE STREET, SUITE 800, NASHVILLE, TN, 37219, US

(73) Assignee: Enamelite, LLC, Clarksville, TN (US)

(21) Appl. No.: 12/849,505

(22) Filed: Aug. 3, 2010

Related U.S. Application Data
(63) Continuation-in-part of application No. 11/142,983, filed on Jun. 2, 2005.
Application No. 11/142,983 is a division of application No. 10/622,090, filed on Jul. 17, 2003, now Pat. No. 7,018,465.

(51) Int. Cl. A61B 5/117; C09D 4/00; B65D 71/00

(52) U.S. Cl. 427/1; 106/285; 206/229

(57) ABSTRACT

An aerosolized fingerprint compositions for spraying onto a surface for the purpose of indentifying latent fingerprints that comprises about 1 to about 75% fingerprint dusting powder and about 25 to about 99% non-CFC propellant.

### PRIORITY INFORMATION

The present application is a continuation-in-part of, and claims benefit of, U.S. application Ser. No. 11/142,983, filed Jun. 2, 2005, which is a divisional application of U.S. application Ser. No. 10/622,090, now U.S. Pat. No. 7,018,465, and claims benefit of U.S. application No. 60/397,664. The contents of all applications are incorporated herein by reference in their entirety.

### FIELD OF THE INVENTION

The present invention relates generally to the fingerprinting art by aiding a person in identifying and “lifting” fingerprints from a surface.

The present invention further relates to an aerosolized fingerprint formulation for aiding a person in identifying and “lifting” fingerprints from a surface.

Accordingly, the spray and method of the present invention increases the efficiency of obtaining a fingerprint while decreasing the amount of time and amount of materials usually required by a ceramist to apply such coatings. The spray of the present invention provides an even coating and consistent quality application.

Additionally, the present invention is advantageous in that clean up is easier and allows for a greater variety of surfaces and angles to be dusted for prints.

### BACKGROUND OF THE INVENTION

Generally, a fingerprint is an impression of the friction ridges of all or any part of the finger. As used herein, “fingerprint” or “print” are used interchangeably, and refer to an impression of a friction ridge. Thus, use of the terms “fingerprint”, or “print” is intended to cover all traditional fingerprints, palm prints, toe prints, etc. A friction ridge is a raised portion of the epidermis on the palmar (palm), digits (fingers and toes), or plantar (sole) skin, consisting of one or more connected ridge units of friction ridge skin. These are sometimes known as “epidermal ridges” which are caused by the underlying interface between the dermal papillae of the dermis and the interpapillary (rete) pegs of the epidermis. These epidermal ridges serve to amplify vibrations triggered when fingertips brush across an uneven surface, better transmitting the signals to sensory nerves involved in fine texture perception. The ridges assist in gripping rough surfaces, as well as smooth wet surfaces.

Fingerprints may be deposited in natural secretions from the eccrine glands present in friction ridge skin (secretions consisting primarily of water) or they may be made by ink or contaminants transferred from the peaks of friction skin ridges to a relatively smooth surface such as a fingerprint card. The term fingerprint normally refers to impressions transferred from the pad on the last joint of fingers and thumbs, though fingerprint cards also typically record portions of lower joint areas of the fingers (which are also used to make identifications).

Fingerprint identification or palm print identification is the process of comparing questioned and known friction skin ridge impressions from fingers or palms or even toes to determine if the impressions are from the same finger or palm. The flexibility of friction ridge skin means that no two finger or palm prints are ever exactly alike (never identical in every detail), even two impressions recorded immediately after each other. Fingerprint identification (also referred to as individualization) occurs when an expert (or an expert computer system operating under threshold scoring rules) determines that two friction ridge impressions originated from the same finger or palm (or toe, sole) to the exclusion of all others.

A known print is the intentional recording of the friction ridges, usually with black printers ink rolled across a contrasting white background, typically a white card. Friction ridges can also be recorded digitally using a technique called Live-Scan. A latent print is the chance reproduction of the friction ridges deposited on the surface of an item. Latent prints are often fragmentary and may require chemical methods, powder, or alternative light sources in order to be visualized.

When friction ridges come in contact with a surface that is receptive to a print, material on the ridges, such as perspiration, oil, grease, ink, etc. can be transferred to the item. The factors which affect friction ridge impressions are numerous, thereby requiring examiners to undergo extensive and objective study in order to be trained to competency. Pliability of the skin, deposition pressure, slippage, the matrix, the surface, and the development medium are just some of the various factors which can cause a latent print to appear differently from the known recording of the same friction ridges. Indeed, the conditions of friction ridge deposition are unique and never duplicated.

There are several types of fingerprint types, including latent prints, patent prints, and plastic prints. For latent prints, although the word latent means hidden or invisible, in modern usage for forensic science the term latent prints means any chance of accidental impression left by friction ridge skin on a surface, regardless of whether it is visible or invisible at the time of deposition. Electronic, chemical and physical processing techniques permit visualization of invisible latent print residue whether they are from natural secretions of the eccrine glands present on friction ridge skin (which produce palmar sweat, consisting primarily of water with various salts and organic compounds in solution), or whether the impression is in a contaminant such as motor oil, blood, paint, ink, etc. There are different types of fingerprint patterns such as an arch, tented arch, a loop, and a whorl. Each indicate what type of fingerprint it is.

Latent prints may exhibit only a small portion of the surface of the finger and may be smudged, distorted, overlapping, or any combination, depending on how they were deposited. For these reasons, latent prints are an “inevitable source of error in making comparisons,” as they generally “contain less clarity, less content, and less undistorted information than a fingerprint taken under controlled conditions, and much, much less detail compared to the actual patterns of ridges and grooves of a finger.”

Patent prints are friction ridge impressions of unknown origins which are obvious to the human eye and are caused by a transfer of foreign material on the finger, onto a surface. Because they are already visible they need no enhancement, and are generally photographed instead of being lifted in the same manner as latent prints. An attempt to preserve the actual print is always made with numerous techniques; for later presentation in court. Finger deposits can include materials such as ink, dirt, or blood onto a surface.

A plastic print is a friction ridge impression from a finger or palm (or toe/foot) deposited in a material that retains the shape of the ridge detail. Commonly encountered examples are melted candle wax, putty removed from the perimeter of window panes and thick grease deposits on car parts. Such prints are already visible and need no enhancement, but investigators must not overlook the potential that invisible latent prints deposited by accomplices may also be on such surfaces. After photographically recording such prints, attempts should be made to develop other non-plastic impressions deposited at natural finger/palm secretions (eccrine gland secretions) or contaminates.

Since the late nineteenth century, fingerprint identification methods have been used by police agencies around the world to identify both suspected criminals as well as the victims of crime. The basis of the traditional fingerprinting technique is simple. The skin on the palmar surface of the hands and feet forms ridges, so-called papillary ridges, in patterns that are unique to each individual and which do not change over time. Even identical twins (who share their DNA) do not have identical fingerprints. Fingerprints on surfaces may be described as patent or latent. Patent fingerprints are left when a substance (such as paint, oil or blood) is transferred from the finger to a surface and are easily photographed without further processing. Latent fingerprints, in contrast, occur when the natural secretions of the skin are deposited on a surface through fingertip contact, and are usually not readily visible. The best way to render latent fingerprints visible, so that they can be photographed, is complex and depends, for example, on the type of surface involved. It is generally necessary to use a ‘developer’, usually a powder or chemical reagent, to produce a high degree of visual contrast between the ridge patterns and the surface on which the fingerprint was left.

Developing agents depend on the presence of organic materials or inorganic salts for their effectiveness although the water deposited may also take a key role. Fingerprints are typically formed from the aqueous based secretions of the eccrine glands of the fingers and palms with additional material from sebaceous glands primarily from the forehead. The latter contamination results from the common human behaviors of touching the face and hair.

The resulting latent fingerprints consist usually of a substantial proportion of water with small traces of amino acids, chlorides, etc., mixed with a fatty, sebaceous component which contains a number of fatty acids, triglycerides, etc. Detection of the small proportion of reactive organic material such as urea and amino acids is far from easy.

Crime scene fingerprints have typically been detected by simple powders, or some chemicals applied at the crime scene. The application of powders for the development of latent fingerprints is one of the earliest known techniques dating back to the nineteenth century. See Advances in Fingerprint Technology, Second Edition, Lee, H. and Gaensslen, R., Eds.; Forensic and Police Science Series; CRC Press: Boca Raton, 2001. The constituents of latent prints facilitate an adherence of powder particles thereby rendering impressions visible. Developed impressions may then be preserved by lifting or photography.

Over the years a number of different methods of powder application have been proposed. The most common method of application for conventional powder dusting is by brushing the surface with appropriate bristled brushes such as animal hair or fiberglass. A major drawback with the typical method is how the powders are packaged and applied. The powders are typically packaged in a jar. The fingerprint brush is dipped into the jar as to allow the powders to accumulate on the brush. They are later transferred from the brush to the surface. This method leads to inaccuracies in actualizing the print and is cumbersome.

Other, less popular, methods have been proposed such as atomizers, sifting, and aerosolized spray, but the results were typically inferior to the standard method of dusting using brushes. See Bridges, B. Practical Fingerprinting. Funk & Wagnalls Company: New York, 1963. Olsen, R. Scott's Fingerprint Mechanics. Charles C Thomas Publisher: Springfield, 1978. Chapel, C. Fingerprinting: A Manual of Identification. Coward McCann, Inc.: New York, 1941.

The atomizer method of applying fingerprint powders consists of blowing the fingerprint powder onto the surface by a blast of air thus reducing the need for physical contact that might destroy or damage the ridge detail. The blast of air from the old style atomizer charged with powder was not in itself strong enough to fully develop an impression and still required brushing to enhance the print. Atomizers had a tendency to paint the surface as the air forced the powder into the surface depressions and never delivered a very even spread of powder. This problem is similar to the previous one in that it is difficult to accurately actualize the print.

The sifting method of applying fingerprint powders consists of applying the powder directly onto the object to be processed and sliding the powder back and forth across the suspected area until enough powder adhered to the latent impressions. A light touchup is still necessary using a fingerprint brush to remove excess powder. This method often resulted in too much powder being applied to the surface thereby destroying or over powdering the print.

Aerosol powders have been tested in the past with inferior results to that of brushing, primarily due to improper air control, clogging of the nozzles causing uneven spray distribution, and poor powder ratios or propellants.

The present invention has many advantageous over prior art methods.

### SUMMARY OF THE INVENTION

It is accordingly one object of the present invention to provide a spray delivery system, including an aerosolized delivery system that will overcome current limitations and disadvantages of lifting fingerprints.

Another object of the present invention is to provide a simple and dependable product and method for efficient and fast developing of latent prints.

Another object of the present invention is to provide a method of lifting prints that is versatile in terms if surfaces, objects, angles, etc.

Another object of the present invention is to provide a spray fingerprint composition that can be used in the process of identifying fingerprints on a surface.

Another object of the present invention is a method of obtaining fingerprints using a spray composition of the present invention.

Another embodiment of the present invention is an aerosolized spray fingerprinting formulation, comprising (weight %): about 1 to about 75% fingerprinting powder; and about 25 to about 99% non-CFC propellant.

Another embodiment of the present invention is a method of obtaining fingerprints, comprising: (1) providing an aerosolized spray fingerprint dusting powder of the present invention; (2) identifying a surface that may contain a latent fingerprint; (3) spraying the surface with the fingerprint dusting powder to actualize latent print; and (4) analyzing actualized print.

Additionally, embodiments of the present invention utilize a dry powder aerosol. Unsuccessful prior art attempts contain a type of binding agent the artificially enhances the ability of the powders to bind to or stick to the sprayed surface. These methods are deficient in that they do not allow the powders to be easily worked to properly expose or develop the finger print because of the nature of the powder. The methods of the present invention comprise a dry powder system/dry propellant system that eliminates the attachment of a sticky layer of powder layer on a surface which would inhibit properly controlling a sprayed powder layer to expose a finger print.

Fingerprints are used to identify an unknown victim, witness, or suspect, to verify records, and most importantly, as links and matches between a suspect and a crime. Occasionally, a print is found that is made with the palm of the hand or a bare foot. These are ordinarily processed by the same methods used for fingerprints. Accordingly, as indicated herein, the term “fingerprint” is generic and is not strictly limited to fingers. It generically includes finger prints, palm prints, toe prints foot prints, and partial prints thereof

Ridges develop on the skin of its fingers and thumbs. These ridges arrange themselves in more or less regular patterns. For purposes of classification, experts divide these ridge patterns into three basic classes: arches, loops, and whorls. When prints are found, an expert compares them with samples.

There are three basic forms of prints: plastic, which are impressions left in soft material like wax, paint, or putty; visible, which are made by blood, dirt, ink, or grease; and latent, which are normally invisible and must be developed before they can be seen and photographed.

The most common way of developing latent prints is by dusting with fingerprint powders. A very fine powder is gently brushed over the surface of an object suspected of having fingerprints. The fine powder sticks to the oils and perspiration that are left behind from the top of the friction ridges of the skin. Great care and skill are required to actualize the latent print. A non-skilled person may cause damage to the ridged line of the fingerprint during the brushing step.

One embodiment of the present invention is an aerosolized fingerprint powder composition, comprising (by weight %): about 1 to about 75% fingerprint dusting powder; and about 25 to about 99% non-CFC dry propellant.

Another embodiment of the present invention is a method of obtaining fingerprints, comprising: (1) providing an aerosolized spray fingerprint powder composition, comprising a fingerprint powder and a dry propellant; (2) providing an isolation device that comprises a small opening defined by at least one wall and a large opening defined by at least one wall; (3) identifying a surface that may contain a latent fingerprint; (4) placing the large opening of the isolation device over the surface; (5) spraying the aerosolized fingerprint powder into the small opening of the isolation device to actualize latent print; and (6) analyzing actualized print.

Another embodiment of the present invention is a kit, comprising: at least one aerosolized container that comprises a fingerprint formulation and a propellant; a finger print brush; an isolation device that is defined by at least one wall and has a fingerprint surface opening and a spray opening.

As indicated herein, aerosol fingerprint powders have been tested in the past with inferior results to that of brushing. Reasons for this inferiority include improper air control, clogging of the nozzles causing uneven spray distribution, and poor powder ratios or propellants. See Olsen, R. Scott's Fingerprint Mechanics. Charles C Thomas Publisher: Springfield, 1978.

Prior art aerosol fingerprint methods did not utilize a portable “isolation device,” which maximizes the effectiveness of the aerosol spray delivery system by capturing the sprayed materials for maximum utilization.

Additionally, with respect to embodiments of the present invention, the powder to propellant ratio of the present invention is greater. Specific examples of the present invention include a formula that utilizes about 1 to 10 ration (powder to propellant).

In practice, the methods of the present invention actualize a print using a low powder concentration. The “low” powder concentration used in embodiments of the present invention unveils a completely new fingerprint revealing processes. Methods use such a small amount of powder with a single burst (less than any prior art method), the powder is basically “layered-on” the print as never before. For example, when using the isolation device of the present invention, and using only 1 spray burst in the top of the isolation device of a black finger print powder, a white substrate, the detection of the black powder not attached to the print with the naked eye is difficult. The method of the present invention typically leaves no “clumps” of powders on the sprayed surface.

To further explain the “layered” technique, with all past and current finger print powder application techniques (by brush, air, aerosol) all apply much more powder than necessary to expose and develop the print. All powder application techniques to date battle inefficiency. Simply stated, with all other know latent powder application techniques, the processes employed requires one to work or move the excess powders off or away from an exposed print, as not to damage or contaminate the print with too much powder. The method of the present invention is the first finger print powder delivery system ever which allows a minimal amount of powders to be applied (maybe less than is required to 100% develop the print) at one single application. With the method of the present invention, if the print is not exposed with the first applied powder amount (normally with a single spray burst) then an additional spray burst may be used to further highlight the print without the fear of “overexposing” the print with too much powder. The method of the present invention is designed to empower the user with a metered approach to powder application. The method of the present invention is highly safe, efficient and predictable for the user who might be a novice. Other powder techniques requires the user to move powders away from the surface, while the method of the present invention allows powders to be safely built up or layered on a surface. This is a much more effective and accurate fingerprint lifting system.

These and other objects will be apparent from the present disclosure and claims. Additional substance, advantages and other novel features of the invention will be set forth in part in the description that follows and in part will become apparent to those skilled in the art upon inspection of the following or may be learned with practice of the invention or improved development. Further, the above embodiments are examples of the present invention and not intended to be limiting thereof

### BRIEF DESCRIPTION OF THE DRAWINGS

FIG. 1 is a drawing that shows the fingerprint formulation of the present invention being sprayed into an isolation device of the present invention, over a surface that is suspected of having a latent print.

FIG. 2 is drawing that shows the same view as FIG. 1, but also providing a cut-away of the isolation device.

FIG. 3 is a side, cut-away view of FIG. 1.

FIG. 4 is a photograph that shows commercial black fingerprint powder under a polarized light microscope at 200 times magnification.

FIG. 5 is a representation of powder from the present invention applied in a method of the present invention.

### DETAILED DESCRIPTION OF THE INVENTION

As stated above, embodiments of the present invention include a spray fingerprint composition that can be used in the process of locating and lifting fingerprints on a surface and a method of obtaining fingerprints from a surface.

The aerosolized spray fingerprint dusting powder compositions of the present invention may be used for obtaining fingerprints. This method comprises identifying a surface that may contain a latent fingerprint; spraying the surface with the fingerprint dusting powder to actualize latent print; and analyzing actualized print. Additionally, the actualized print may be documented by photographing the actualized print or removing the actualized print with an adhesive material.

Glass Frit

One optional component of the present invention is glass frit particles. Without being bound by theory or mechanism, glass frit, when used, may impart enhanced finger print ridge detail and clarity. Glass frit usable in connection with the present invention is available from many different manufacturers worldwide. In embodiments of the present invention, the same glass frit currently used in the dental profession and hobby ceramist practicing the brush technique may be used in the formulation.

The glass frit may be in the form of either a natural or man-made mixture of inorganic chemical substances. The glass frit is produced by rapidly quenching a molten, complex amalgamation of materials. Such glass frit is available from a number of manufacturers under varying designations.

In embodiments, at least about 90% of the frit have a particle size of about 25 microns and under. In another embodiment, at least about 90% of the frit have a particle size of about 20 microns and under. In another embodiment, at least about 75% of the frit have a particle size of about 15 microns and under. In another embodiment, at least about 90% of the frit have a particle size of about 15 microns and under. In another embodiment, at least about 75% of the frit have a particle size of about 10 microns and under. In another embodiment, at least about 90% of the frit have a particle size of about 10 microns and under. Further, in embodiments of the present invention, at least about 75% of the frit have a particle size of about 8 microns and under. In another embodiment, at least about 90% of the frit have a particle size of about 8 microns and under.

One source of glass frit is the Ferro Corporation, Coating Division, located in Cleveland, Ohio. 3227 leadless frit available from Ferro is an example of glass frit of the present invention. Further, the frit can be milled such as ball milled or jet milled in a ceramic mill to arrive at the preferred particle size.

Because of varying percentages of chemical components in the various glass frit formulations available from manufacturers, the intrinsic characteristics of the glass frit also vary. As a result, the particular weight percentages of the individual components of the glaze composition may need to be adjusted across the range set forth above to provide proper and consistent results.

In examples of the present invention, the glass frit is present in amounts ranging from about 4 to about 50 weight % of the total composition. In other examples, this glass frit is present in an amount of from about 8 to about 35%. In other examples, this range is from about 9 to about 17%.

Propellant

Prior art compositions for aerosol applications typically used, as a suspension agent, chlorofluorocarbons, which are now known to be hazardous. Examples include 1,1,1-trichloroethane and Freon TF 22 propellants. Inhalation or swallowing vapors may irritate the respiratory tract and affect the central nervous system. Over exposure symptoms include headache, dizziness, weakness, and nausea. Higher levels of exposure (>5000 ppm) can cause irregular heartbeat, liver, and kidney damage, fall in blood pressure, cardiovascular damage, unconsciousness and even death. 1,1,1-trichloroethane is also thought by some to be a possible carcinogen. Furthermore, CFC materials are the source of a myriad of environmental problems, including adversely affecting the ozone layer. Therefore, their use is not unacceptable in the fingerprinting formulations of the present invention. Accordingly, one advantage of the present invention is the non-CFC propellant.

The propellant used in connection with the present invention is a dry propellant. A dry propellant contains no additives that are added to bind or artificially adhere sprayed powder materials to each other or to a surface. The dry propellants of the present invention are hydrocarbon-based or compressed gas materials such as CO₂, propane, butane or any derivative thereof, or combinations thereof.

The propellant used in connection with the present invention is also a non-CFC propellant. One propellant that may be used is a hydrocarbon propellant. Further examples include isobutane, butane or any mixtures thereof While spray pressures may range between about 17-132 psig, for best results and the most consistent spray characteristics, the compositions of the present invention are packaged at a pressure in the range of between about 17-56 psig.

The butane and isobutane hydrocarbon propellants are available, for example, from Aeropres Corporation, Shreveport, La. under the designation of A-17, A-31 up to the strongest pressure of A-132 propellants.

The non-CFC propellant of the present invention may be present in the composition in amounts ranging anywhere from about 10-99%, about 50-99%, about 70-96%, about 80-93%, and/or about 85-92% by weight of the total composition.

### Fingerprint Powder and Formulation

The fingerprint powder of the present invention is not known to be critical. That is, any fingerprint powder known in the art is capable of being used with the present invention. For example, the fingerprinting powder may be selected from the group consisting of non-metallic and non-fluorescent powders commonly used that can be colored black, white, silver, grey, red, etc., or combinations thereof Fluorescent powders commonly used care colored red, green, yellow, orange, and blue. Metallic powders commonly used are colored silver, copper, red, orange, green, blue, yellow, etc., or combinations thereof In embodiments, some types of latent print powders are comprised of a combination of materials to maximize the detection of latent fingerprints.

Powders of varying color are used to get the maximum contrast with the background material. The excess powder is blown off, leaving a clear impression from the powder that adheres to the ridges of the print. The print can then be photographed and lifted with an adhesive material such as tape.

The powders of the present invention may be comprises of various materials, including, for example, talc, silica, barium sulfate, calcium carbonate, gypsum, alumina, agalmatolite, lithopone, zinc oxide, silicon oxide, titanium oxide, carbon black, graphite, molybdenum disulfide, iron oxide, silica black, chrome black, mineral black, vine black, bone black, silicon carbonate, and mixtures thereof

The following is a non-limiting list of powders that can be used in connection with the present invention. Like all examples presented herein, it is presented for exemplary purposes, and is not to be construed as being limiting of the present invention.

### A) Traditional Powders

| Powder Type | Ingredient | CAS# | Percent |
|---|---|---|---|
| Lighting powders | | | |
| White | Titanium Dioxide | 13463-67-7 | <45 |
| | Zinc Stearate | 557-05-1 | 30 |
| | Lycopodium | 8023-70-9 | >10 |
| BiChromatic | Alumimum | 7429-90-5 | 9.5 |
| | Carbon Black | 1333-86-4 | 10 |
| Silver-Grey | Alumimun | 7429-90-5 | 19 |
| Sirchie powders: | | | |
| Hi-Fi Black Volcano, Silk | Carbon Black | 1333-8634 | ND |
| | Lycopodium | 0 | ND |
| Hi-Fi Black Volcano, Silk | Black Iron Oxide | 1317-61-9 | 36.5 |
| Carbon Free | Gum Arabic | 9000-01-5 | 54.5 |
| | Lycopodium | 0 | 9 |
| OPTI Black-T | Carbon Black | 1333-86-4 | 75 |
| | Talc | 14807966 | 25 |
| OPTI Black (Pumice) | Carbon Black | 1333-86-4 | 75 |
| | Pumice | 1332-09-8 | 25 |
| Hi-Fi Volcano Red | Gum Arabic | 9000-01-5 | >16 |
| | Lycopodium | 0 | >16 |
| | Pigment red 48:1 | 7585-41-3 | >65 |
| Hi-Fi Volcano Silk Grey | Aluminum | 7429-90-5 | 15 |
| | Lycopodium | 0 | 35 |
| | Titanium Dioxide | 13463677 | 40-49 |
| Hi-Fi Volcano White | Titanium Dioxide | 13463677 | 90 |
| | Zinc Stearate | 557051 | 10 |

### B) Flourecent Powders

| Powder Type | Ingredient | CAS# | Percent |
|---|---|---|---|
| Lighting powders (their MSDS only lists hazardous ingredients): | | | |
| Redwop Powder | Extender | Proprietary | 80-90 |
| | Dyed Polymer | Proprietary | 10-20 |
| Greenwop Powder | Extender | Proprietary | 80 |
| | Dyed Polymer | Proprietary | 20 |

### C) Metallic Powders

| Powder Type | Ingredient | CAS# | Percent |
|---|---|---|---|
| Sirchie powders: | | | |
| Hi-Fi-Volcano, Silver | Aluminum | 7429-90-5 | 80 |
| | Lycopodium | 0 | 20 |
| Hi-Fi Volcano, Copper | Copper | 7440-50-8 | 14 |
| | Gum Arabic | 9000-01-5 | 58 |
| | Lycopodium | 0 | 14 |
| | Titanium Dioxide | 13463677 | 14 |

### D) Flourecent Magnetic Powders (FMP)

| Powder Type | Ingredient | CAS# | Percent |
|---|---|---|---|
| Sirchie powders: | | | |
| REDcharge FMP | Iron | 7439-89-6 | 49-49.5 |
| | Lycopodium | 0 | 25 |
| | Red AX pigment | 0 | 25 |
| ORANGE charge FMP | Yellow AX Pigment | 0 | 25 |
| | Iron | 7439-89-6 | 46 |
| | Lycopodium | 0 | 25 |
| | Titanium Dioxide | 13463677 | 4 |
| GREEN charge FMP | Iron | 7439-89-6 | 46 |
| | Lycopodium | 0 | 25 |
| | Green AX Pigment | 0 | 25 |
| | Titanium Dioxide | 13463677 | 4 |
| BLUE charge FMP | Blue T Pigment | 0 | <20 |
| | Iron | 7439-89-6 | <40.5 |
| | Lycopodium | 0 | 22 |
| | Phthalocyanine blue | 147148 | >13 |
| | Titanium Oxide | 13463677 | >3.5 |
| DAZZLE Orange FMP | Yellow AX pigment | 0 | >11 |
| | Magnetite | 1317619 | <89 |
| DAZZLE Red FMP | Magneitie | 1317619 | <89 |
| | Rec AX pigment | 0 | >11 |
| DAZZLE Yellow FMP | Magnetite | 1317619 | <89 |
| | Yellow AX pigment | 0 | >11 |

Particle Sizes

Particle sizes of the fingerprint powder can vary. Typically particle sizes range from about 1 to about 50 microns. In some embodiments, about 90% of the particles are less than about 50 microns. In other embodiments, at least about 75% of the particles are less than about 30 microns. In other embodiments, at least about 50% of the particles are less than about 25 microns. In other embodiments, at least about 25% of the particles are less than about 25 microns. In other embodiments, at least about 25% of the particles are less than about 15 microns. In other embodiments, at least about 10% of the particles are less than about 25 microns. In other embodiments, at least about 75% of the particles are less than about 25 microns.

The table below lists examples of particle sizes and particle size distribution for typical print powders (in microns):

| Manufacturer | Powder Color | <10% | <25% | <50% | <75% | <90% |
|---|---|---|---|---|---|---|
| Evident | Black | 5.7 | 11.18 | 23.62 | 26.49 | 27.89 |
| Sirchie | Black | 24.13 | 25.39 | 26.62 | 28.26 | 33.18 |
| Sirchie | Grey | NA | 10.71 | 20.61 | 27.86 | 29.68 |
| Lightning | Silver/Grey | 5.7 | 8.2 | 15.40 | 28.17 | 45.77 |
| Evident | White | 2.7 | 4.64 | 9.69 | 17.44 | 24.87 |
| Lightning | White | 1.83 | 2.37 | 3.44 | 5.65 | 9.32 |
| Lightning | RedWop | 24.73 | 26.59 | 27.72 | 28.79 | 29.65 |

Various rations, powder to propellant, can be used. For instance, an example of an aerosolized spray fingerprinting formulation of the present invention comprises (weight %): about 1 to about 75% fingerprinting powder; and about 25 to about 99% non-CFC propellant. In other examples, the fingerprinting powder can be present in any amount between about 1 and about 50%; between about 4 and about 30%; between about 5 and about 15%; or between about 9 and about 12%.

In other examples, the propellant can range from about 50 to about 99%; about 70 to about 96%; about 80 to about 93%; or about 85 to about 92%.

In one embodiment, the ratio is, powder to propellant, 1 to 5. In another embodiment, the ratio is 1 to 6. In another embodiment, the ratio is 1 to 7. In another embodiment, the ratio is 1 to 8. In another embodiment, the ratio is 1 to 9. In another embodiment, the ratio is 1 to 10. In another embodiment, the ratio is 1 to 11. In another embodiment, the ratio is 1 to 12. In another embodiment, the ratio is 1 to 13. In another embodiment, the ratio is 1 to 14. In another embodiment, the ratio is 1 to 15. In another embodiment, the ratio is 1 to 16. In another embodiment, the ratio is 1 to 17. In another embodiment, the ratio is 1 to 18. In another embodiment, the ratio is 1 to19. In another embodiment, the ratio is 1 to 20. In another embodiment, the ratio is 1 to 21. In another embodiment, the ratio is 1 to 22. In another embodiment, the ratio is 1 to 23. In another embodiment, the ratio is 1 to 24. In another embodiment, the ratio is 1 to 25.

Isolation Device

Embodiments of the present invention comprise the use of an isolation device. The isolation device is used to control and direct the misting of the aerosolized fingerprint particles.

In embodiments of the invention, the isolation device can be foldable to a collapsed position for convenient handling and storage.

The nature of the material from which the isolation device may be made is not known to be critical. In embodiments, it is made from any suitable inexpensive, durable, light-weight material, such as a polymeric material or paperboard, or the like, so that it may be discarded.

The isolation device is typically conical and hollow, with the aerosol spray being discharged into the small opening and the larger opening enveloping the surface to be fingerprinted. The isolation device of the present invention does not have to form an air-tight seal over the surface, it just simply directs the mist to quickly and efficiently cover and build up the print.

The isolation device provides a chamber-like atmosphere that allows the vapors to mist and swirl around the surface to be printed. As indicated herein, the methods of the present invention allow for very little powder to be used. One advantage of this feature is that the method leaves very little space to be cleaned.

The isolation device can have a square base, round base, oval base, etc. As stated herein, the isolation device can be foldable or collapsible so that it can be easily transported, concealed, etc.

U.S. Pat. No. 6,299,674 to Takamuru et al., incorporated herein by reference, generally discusses fingerprinting methods and discloses a fingerprint detecting agent and method which can be used to detect latent fingerprints being in a wet condition.

U.S. Pat. No. 4,176,205 to Molina, incorporated herein by reference, generally discusses fingerprinting methods and discloses a fingerprint powder and a method for developing latent prints. The fingerprint powder of Molina can be applied by blowing the powder over a surface containing latent prints, or by brushing by pouring the powder on such surface to reveal a print that can be photographed or lifted by applying tape or a strippable coating over the print. A single spray burst from the aerosol can meters the amount of powder dispensed empowering the user to control the amount of powder necessary to detect latent fingerprints. Additional spray bursts can be employed to further build up powder to further expose a latent fingerprint in a predictable and user-friendly manner lessoning the chance of damaging the print.

The fingerprint spray formulation of the present invention has at least two advantages in that it allows latent prints to be actualized with greater ease and with as little damage as possible to the print.

Without being bound by theory or mechanism, one advantage of the present invention when compared to traditional fingerprinting is that the present invention more evenly disperses fingerprinting powder.

Additionally, another advantage of the present invention is the use of embodiments thereof to provide a coating on a surface to indicate where there is a high probability of the location of the print. This step can be followed with any required brushing to fully expose the print for lifting. Thus, an advantage is the ability to have a controlled amount of powders to be dispensed. In certain embodiments, only one blast of a second or less is the engineered calculated amount of powder necessary to expose and optimize the print, the can limits the amount of powders present which will limit the chance an novice finger print user would overexpose powders to the surface which could over stimulate the print or even damage the print potentially beyond repair. Further, excess brushing has the potential to damage the print.

Additional embodiments of the present invention include its use in an “isolation device.” The isolation device is essentially a chamber (such as a poster tube, for example, that may be about 1 to 2 inches opening at the top and about 6 to 8 inches open at the bottom), one spray burst at the top of the tube will dispense enough powders to completely dry powder coat an object (drinking glass, knife, gun, bullet casing etc . . . ). The tube is placed over an object or objects, one spray burst is sprayed into the top, the powder swirls inside the tube and is allowed to settle for about 5 to 10 seconds . . . then removed, and the objects are then coated ready for the final act which is the brushing to fully expose the print for lifting or photography.

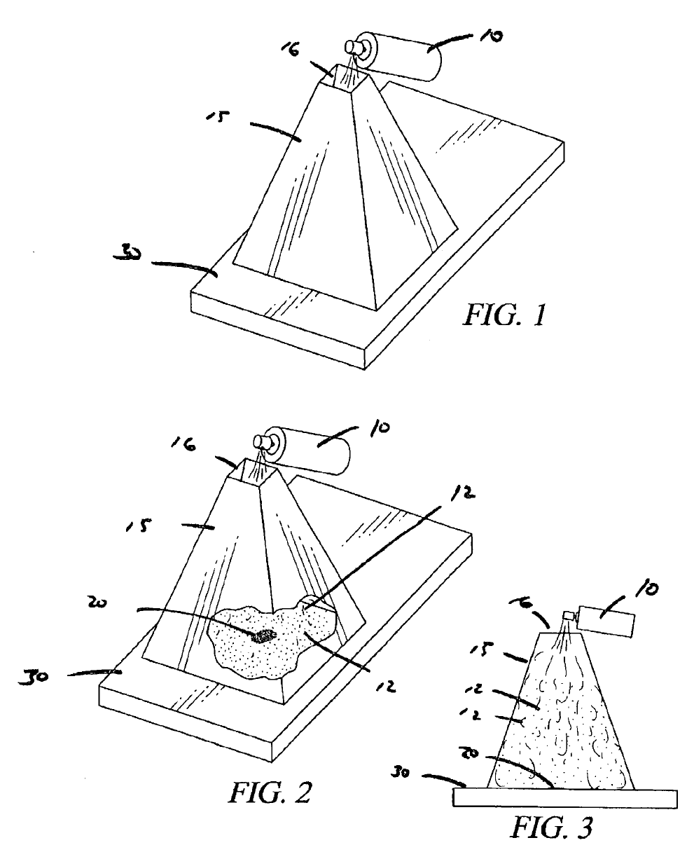

FIGS. 1-3 show an embodiment of the present invention. The aerosolized fingerprint formulation of the present invention 10 is sprayed into an opening 16 of the isolation device 15. The isolation device is placed over a surface 30 that is suspected of comprising a latent print 20. Of course, the surface can be on an object such as a glass, plate, gun, etc. The particles 12 swirl in the isolation device and help build the print.

### Examples

The following examples are presented to further illustrate the invention. But, it should be recognized that the invention is not to be considered limited thereto.

### Example 1

This Example is a preferred fingerprint composition. About 2 grams of Lightning White™ fingerprint powder is placed in an aerosol can manufactured by CCL Container Corporation, Hermitage, Pa. The container is provided with two steel mixing balls that are about 4 to 6 mm in diameter. A dip tube is asserted in the can and them the can is crimped and sealed. An aerosol valve is supplied by Summit Packaging Systems, Inc., Manchester, N.H. In this example, the valve assembly comprises an actuator (Part. No. 78858), stem (920103), stem gasket (77505), spring (77401), body (97311), dip tube (200610), and mounting cup (77792). The crimped aerosol can is then charged with a hydrocarbon butane propellant to a pressure of about 31 psig at 70 degrees Fahrenheit. The product is actualized with 20 grams of A-31 propellant. The final actuator button is fixed and the product is ready for spraying.

### Example 2

This Example demonstrates four exemplary embodiments of the present invention. These non-limiting example incorporate four different aerosol powders using various can sizes filled with particular powder masses (small can: 1 g powder, medium can: 4 g powder, large can: 7 g powder). Two different non-porous substrates bearing latent print impressions from an oil standard of varying deposition intensity and ages were used in this example. Half of the impressions were processed using cyanoacrylate ester (CNA) prior to the application of the powder. The other half of the impressions were processed directly with the aerosol powder fingerprint formulations of the present invention. Results indicate this process to be an effective technique on non-porous surfaces without CNA. In summary, this example shows that the present invention is an effective fingerprinting method, and that it maintains a relatively even distribution of powder, controls the amount of powder deposited, and decreases most of the brush contact with the surface thereby lessening the chances of damage to impressions

Materials and Methods: Four commercial fingerprint powders of the most readily used brands (Sirchie, Lightning, Peavey, Tri-Tech, and Evident) are prepared in aerosol cans of various sizes (small, medium, and large) and filled with particular powder masses, 1 g, 4 g, and 7 g, respectively, maintaining a similar powder to propellant (about 10 to about 1)

The environment for this example comprised a temperature and humidity of about 72.5 degrees Fahrenheit and 21%, respectively.

A commercial sebaceous control matrix standard (Armor Forensics #1-2792) was used for the deposition of the latent print impressions on two types of non-porous substrates—smooth and textured plastic. Thirty-six latent print impressions were deposited on each type of substrate within each particular deposition age—zero days, seven days, and fourteen days. The depositions consisted of three successive latent print impressions decreasing in deposition intensity (amount of matrix available for deposition) for a total of twelve gradients. An additional twelve gradients were processed with CNA prior to the application of powders. Each of the twenty-four gradients (twelve gradients processed with CNA and twelve gradients without) were subject to development by each of the four particular powders distinguished by the three aerosol can sizes containing a particular powder mass per can.

The application of the aerosolized powders for each of the latent print impressions was as follows: (a) the isolation device, a small cone with openings at each end, was placed over the area of interest containing the impression to retain the powder in the desired area of application; (b) each aerosol can was thoroughly shaken for 10-15 seconds to maintain the powder consistency and spray distribution; (c) each aerosol can was directed into the isolation device and a single burst was sprayed for approximately ⅓ second up to about 1 second; (d) the powder was allowed to settle for approximately 10 seconds prior to removing the isolation device; and (f) the area of interest was lightly dusted with a fiberglass brush (Evident #1008) to remove excess powder deposition.

All latent print impressions developed were digitally captured (Nikon D2Xs) prior to a visual analysis of the printed images (Xerox Phaser 7750DN PS) by nine certified latent print examiners. Under single blind procedures each certified latent print examiner judged the developmental quality of each of the 432 latent print impressions according to the clarity of the developed impression by assigning a numerical rating on a scale from zero to five based on the following: 0=No development; 1=Poor development; ridge structure unclear; 2=First level detail—visible pattern type with unclear ridge path configurations; 3=Second level detail present—visible ridge path configurations; 4=Good development—clear and distinct ridge path configurations; 5=Excellent development—clear ridge path configurations with distinct ridge and pore structure.

Additionally, each examiner was provided with the following definition for clarity: “Clearness, i.e., how well friction skin detail is recorded in a print. In other words, how well the details from 3-D ridges are reproduced in the 2-D print is referred to as the clarity of the print. When most of the detail found on the friction ridges is reproduced in the friction ridge print, the print is considered clear. If few of the details from the friction ridges are reproduced, the print is considered unclear.” See Ashbaugh, D. Quantitative-Qualitative Friction Ridge Analysis. CRC Press. 1999.

All images were presented to each examiner in a format so that only the study coordinators were aware of the image identity. Data was recorded and plotted in a format similar to the answer sheets provided to each examiner for interpretation.

Results: The ratings given by the examiners for each image were averaged thereby revealing an overall rating for each image that can be related back to a level of development listed on the rating scale. These averaged ratings were used to compare substrate types, powder colors, prior processing, deposition intensity, can size, and age. Impressions receiving an average rating of 3.0 or higher are considered to be suitable for comparative purposes.

### Substrate Type

An overall comparison of the developmental quality of the impressions on smooth versus textured surfaces for each specific age of impression is illustrated in Table 1. These results indicate no significant difference in the overall rating for those impressions developed on either substrate for each age. It was noted, however, that Day zero impressions on both surfaces were equivalent in comparative value whereas Days seven and fourteen, while similar in their overall rating, were not suitable for comparative purposes.

TABLE 1
An overall comparison of the developmental quality of the impressions
on smooth versus textured surfaces for each specific age.

| | Smooth | Textured |
|---|---|---|
| Day 0 | 3.3 | 3.4 |
| Day 7 | 2.8 | 2.5 |
| Day 14 | 2.6 | 2.2 |

### Powder Colors

Table 2 concentrates on the ratings of the individual powders used and the effect of age on their developmental capabilities. Based on this information, it can be quickly gathered that the black powder was less effective in all ages when compared to the grey, white, and fluorescent powders. The highest quality of development came from both the grey and white powders presenting relatively equal quality ratings for all ages—both of which were suitable for comparative purposes for all ages, except Day fourteen, although close in proximity did not meet the threshold. The fluorescent powder followed in developmental quality behind white and grey powders, while still meeting the threshold but only on Day zero, Day seven impressions fell just under the threshold while Day fourteen impressions were not suitable for comparison.

TABLE 2
An overall comparison of the developmental quality of the impressions
developed with various powders for each specific age.

| | Grey | White | Black | Fluorescent |
|---|---|---|---|---|
| Day 0 | 4.0 | 4.2 | 2.1 | 3.1 |
| Day 7 | 3.2 | 3.4 | 1.3 | 2.8 |
| Day 14 | 2.9 | 2.9 | 1.3 | 2.5 |

### Prior Processing

With relation to the impressions processed with CNA and those that were not, Table 3 demonstrates a comparison of the overall ratings between those impressions. More specifically, impressions not processed with CNA prior to powder application maintained a more consistent developmental quality throughout all ages, whereas those that were processed with CNA exhibited a considerable drop in quality past Day zero resulting in development too poor for comparative purposes.

TABLE 3
An overall comparison of the developmental quality of those impressions
previously processed with CNA versus those that were not.

| | CNA | Non-CNA |
|---|---|---|
| Day 0 | 3.0 | 3.6 |
| Day 7 | 1.9 | 3.4 |
| Day 14 | 1.6 | 3.2 |

### Deposition Intensity

For each of the three successive deposition intensities per age, Table 4 breaks down the combined ratings for each deposition. Overall, the development of each deposition intensity differed very little between ages, suggesting age had little to no effect on developmental capabilities. Deposition one impressions were superior in quality by meeting the threshold for comparative purposes; however, depositions two and three impressions did not develop to a comparable degree across all ages.

TABLE 4
An overall comparison of the developmental quality on successive
deposition intensities (amount of matrix available for development).

| | Deposition 1 | Deposition 2 | Deposition 3 |
|---|---|---|---|
| Day 0 | 3.5 | 2.7 | 2.5 |
| Day 7 | 3.4 | 2.8 | 2.4 |
| Day 14 | 3.1 | 2.6 | 2.2 |

### Can Size

A comparison of the quality of the impressions developed using various can sizes filled with particular powder masses (small can: 1 g powder, medium can: 4 g powder, and large can: 7 g powder) for each particular powder is illustrated in Tables 5 through 7 distinguished by age. These results indicate that for the majority of the cases difference in the can sizes and powder masses did not affect the quality of development. Interestingly for fluorescent powder, however, for both Days seven and fourteen, there was a noticeable difference in the developmental quality between the small can and the large can to such a degree that only those impressions developed with the large can were suitable for comparative purposes.

TABLE 5
An overall comparison of the developmental quality of the impressions
on Day zero using various can sizes for each particular powder.

| | Small Can | Medium Can | Large Can |
|---|---|---|---|
| | (1 g powder) | (4 g Powder) | (7 g Powder) |
| Grey | 3.9 | 4.2 | 3.9 |
| White | 4.3 | 4.1 | 4.1 |
| Black | 2.1 | 2.0 | 2.0 |
| Fluorescent | 3.0 | 3.2 | 3.1 |

TABLE 6
An overall comparison of the developmental quality of the impressions
on Day seven using various can sizes for each particular powder.

| | Small Can | Medium Can | Large Can |
|---|---|---|---|
| | (1 g powder) | (4 g Powder) | (7 g Powder) |
| Grey | 3.0 | 3.1 | 3.4 |
| White | 3.2 | 3.3 | 3.5 |
| Black | 1.4 | 1.2 | 1.5 |
| Fluorescent | 2.3 | 2.9 | 3.2 |

TABLE 7
An overall comparison of the developmental quality of the impressions
on Day fourteen using various can sizes for each particular powder.

| | Small Can | Medium Can | Large Can |
|---|---|---|---|
| | (1 g powder) | (4 g Powder) | (7 g Powder) |
| Grey | 2.8 | 2.9 | 3.1 |
| White | 2.5 | 3.2 | 3.0 |
| Black | 1.0 | 1.1 | 1.6 |
| Fluorescent | 1.7 | 2.5 | 3.4 |

### Deposition Age

A comparison of the quality of the impressions developed at different ages indicates decreasing developmental quality as the age of the impression increases. Overall, Day zero impressions were suitable for comparative purposes having an average rating of 3.3, whereas Days seven and fourteen impressions were not, having a rating of 2.7 and 2.4, respectively.

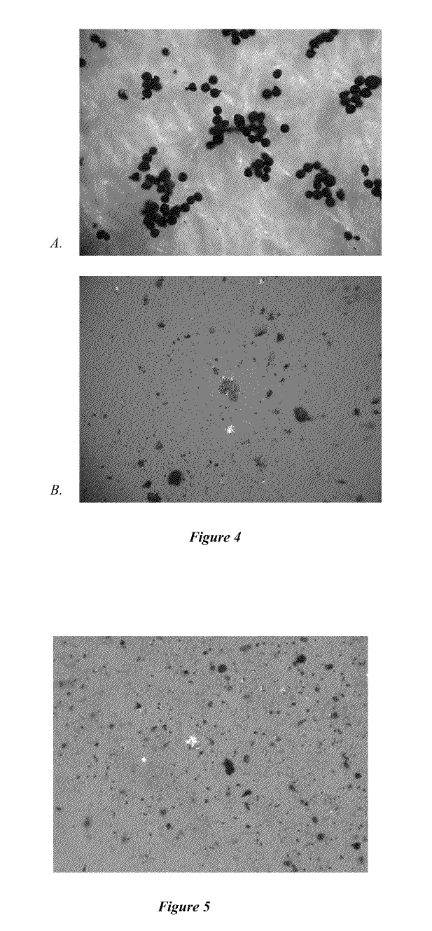

In relation to the developmental capabilities of the different powders used in this study, it is interesting to note the difference in the quality of the impressions developed with the black powder as compared to the white and grey powders. This may be attributable to the brands of powders used or the powder composition and morphology of the individual particles which is illustrated in FIGS. 4 and 5. In addition to the observations of the black powder, the fluorescent powder exhibited moderate development overall that may be explained, in part, because the powder had a tendency to create speckling in and around the furrows of the impression which may have contributed to the inferior quality observed in relation to the white and grey powders.

The effect of CNA processing prior to the application of the powders resulted in little to no development at times, whereas those impressions of the same age with no prior processing maintained discernible ridge detail. These results may be related to the age of the impressions having a possible negative affect on CNA development, which then, may have affected the powders' ability to develop those impressions. Based on this observation, it appears that this method would not be conducive for use in combination with CNA. Further studies are warranted as to why these results were observed.

Multiple deposition intensities were evaluated to ascertain the sensitivity of the powders to develop quality impressions. The quality of development declined for each successive deposition intensity culminating with deposition three (least amount of matrix available for development) maintaining a moderate level of quality development by having an average rating of 2.4 for all ages combined.

Although three separate powder masses were tested, each from a different can size, these variables did not contribute to a significant difference in the developmental quality, probably due to the fact that all cans contained a similar powder to propellant ratio. However, the differences in the developmental quality of fluorescent powder, as noted in Tables 6 and 7, between the small and large cans are interesting, but warrant further studies with particular emphasis on the possible factors which may have contributed to this result.

While the age of the impression did seem to have an affect on the powders' ability to develop the impressions, Day fourteen impressions were still developed with a moderate level of quality having an average rating of 2.4. From this observation it would appear this method would not be precluded from use on impressions exposed for a couple of weeks; however, it should be cautioned that normal environmental conditions may differ to a considerable degree when compared to the controlled laboratory settings of this study.

As shown herein, the methods of the present invention are a simple and convenient process that was very effective in controlling the amount of powder deposited on the substrate surface allowing for the powder to be evenly distributed. Additionally, the present invention is a significant improvement over the prior art.

When the initial spray did not immediately develop the impressions, the powders did reveal an outline of the impressions which could then be fully developed after a light brushing using a clean fiberglass fingerprint brush each time. By using the isolation device, the powder was effectively contained while allowing it to settle around the area of interest, which also cuts down on health risks of airborne powders, cleanup, etc. Additionally, the present invention is an effective and less challenging technique, especially for the inexperienced. It helped by maintaining a relatively even distribution of spray, controlling the amount of powder deposited, and decreasing brush contact with the surface thereby lessening the chances of damage to the impression.

As described above, numerous benefits will result from employing the concepts of the present invention composition.

The invention thus being described, it would be obvious that the same may be varied in many ways. Such variations should not be regarded as a departure from the spirit and scope of the present invention, and all such variations as would be obvious to one of ordinary skill in the art are intended to be included within the scope of the following claims.

This application references various patents and/or publications. All such patents and/or publications are expressly incorporated herein by reference in their entirely.

Finally, unless otherwise indicated, all numbers expressing quantities of ingredients, reaction conditions, and so forth as used in the specification and claims are to be understood as being modified by the term “about.” Accordingly, unless specifically indicated to the contrary, the numerical parameters set forth in this specification and claims are approximations that may vary depending on the desired properties sought to be obtained by the present invention.

Notwithstanding that the numerical ranges and parameters setting forth the broad scope of the inventions are approximations, numerical values set forth in the specific examples are reported as precisely as possible.

We claim:

1. An aerosolized fingerprint powder composition, comprising (by weight %):
about 1 to about 75% fingerprint dusting powder; and
about 25 to about 99% non-CFC dry propellant.

2. The composition of claim 1, wherein at least about 75% of the fingerprint powder has a particle size of about 30 microns or less.

3. The composition of claim 1, wherein at least about 95% of the fingerprint powder has a particle size of about 50 microns or less.

4. The composition of claim 1, in combination with a pressurized container, wherein the fingerprint powder and the propellant are stored in the can under pressure.

5. The composition of claim 1, wherein the fingerprint powder is a metallic fingerprint powder.

6. The composition of claim 1, consisting essentially of the propellant and the fingerprint powder.

7. The composition of claim 1, comprising about 1 to about 50 weight % fingerprint powder.

8. The composition of claim 1, comprising about 50 to about 96 weight % propellant.

9. The composition of claim 1, wherein the non-CFC propellant is a hydrocarbon propellant.

10. The composition of claim 9, wherein the hydrocarbon propellant is selected from the group consisting of isobutene, butane, and mixtures thereof

11. The composition of claim 1, with a ratio of about 1 to 10, powder to propellant.

12. The composition of claim 1, with a ratio of from about 1 to 5, powder to propellant to about 1 to 25, powder to propellant.

13. A method of obtaining fingerprints, comprising:
(1) providing an aerosolized spray fingerprint powder composition, comprising a fingerprint powder and a dry propellent;
(2) providing an isolation device that comprises a small opening defined by at least one wall and a large opening defined by at least one wall;
(3) identifying a surface that may contain a latent fingerprint;
(4) placing the large opening of the isolation device over the surface;
(5) spraying the aerosolized fingerprint powder into the small opening of the isolation device to actualize latent print; and
(6) analyzing actualized print.

14. The method of claim 13, wherein the isolation device is conical.

15. The method of claim 13, wherein after the spraying step, the small opening of the isolation device is covered to allow the particles to swirl around the print and attach to the print.

16. A kit, comprising:
at least one aerosolized container that comprises a fingerprint formulation and a propellant;
a finger print brush;
an isolation device that is defined by at least one wall and has a fingerprint surface opening and a spray opening.

17. The kit of claim 16, wherein the isolation device is foldable into a flat form.
